(19) United States
(12) Patent Application Publication
(10) Pub. No.: US 2003/0171397 A1
(43) Pub. Date: Sep. 11, 2003

(54) Potentiation of antineoplastic agents using sigma-2

(76) Inventors: Keith W. Crawford, S.E. Washington, DC (US); Wayne D. Bowen, Bethesda, MD (US)

Correspondence Address: KLARQUIST SPARKMAN, LLP, 121 SW SALMON STREET, SUITE 1600, PORTLAND, OR, 97204, US

(21) Appl. No.: 10/275,830

(22) PCT Filed: May 8, 2001
(86) PCT No.: PCT/US01/40688

(51) Int. Cl. A61K 31/45; A61K 31/46

(52) U.S. Cl. 514/304

(57) ABSTRACT

The present invention relates to the use of sigma-2 agonists to potentiate the activity of antineoplastic agents. These substances are useful for treating cancerous tumors and, in particular, drug resistant tumors in humans. Methods for sensitizing multidrug resistant cells to antitumor agents comprising contacting the cells with a sigma-2 agonist are also described.

### TECHNICAL FIELD OF THE INVENTION

This invention relates to a method of antineoplastic therapy effective in treating both resistant and non-resistant tumors.

### BACKGROUND OF THE INVENTION

Sigma receptors are high affinity binding sites for several classes of drugs with psychotropic activity. Due to high affinity for most of the typical neuroleptics such as haloperidol, much of the initial interest in sigma receptors was driven by their potential role in the actions of antipsychotic drugs. However, the presence of sigma receptors in many tissues outside of the nervous system suggests that they subserve a more general role than a neurotransmitter receptor. Two subclasses of sigma receptors, sigma-1 and sigma-2 receptors, have been identified. Both subtypes of sigma receptor are expressed in very high density (hundreds of thousands to millions per cell) in tumor cell lines derived from various tissues. These include neuroblastomas, glioma, melanoma, prostate, breast and lung carcinomas. Exposure of tumor cell lines or primary cultures of rat neuronal cells to sigma receptor ligands, including haloperidol and its major metabolite reduced haloperidol, resulted in cell morphological changes which included loss of processes, rounding, and detachment from substratum. Continued exposure resulted in cell death. The pharmacological profile was consistent with mediation by sigma-2 receptors. This was indicated by the activity of the sigma-2 selective compounds CB-64D and ibogaine and lack of activity of sigma-1 selective (+)-benzomorphans such as (+)-pentazocine.

Sigma-2 agonists cause phosphatidyl serine translocation, DNA fragmentation, and chromatin condensation, indicating that sigma-2 receptors induce apoptosis. The apoptotic effect of sigma ligands was demonstrated in both neuronal-type cells and in a number of tumor cell types. These include prostate carcinoma and breast carcinoma. Sigma-2 agonists appear to induce apoptosis by an unusual mechanism. Mutations in the tumor suppresser gene, p53, are the most frequently observed genetic aberrations in tumors, occurring in up to 50% of some tumor types. In tumor cells with p53 mutations, a diminished response to agents that induce apoptosis has been observed (Ryan and Vousden, 1998; Lomax et al., 1998), and these tumors may be clinically-resistant to antineoplastic drugs that produce DNA-damage-,such as actinomycin-D and doxorubicin (Wallace-Brodeur and Lowe, 1999). Sigma-2 receptor ligands, including CB-64D, were found to induce apoptosis in breast tumor cell lines possessing p53 mutations, whereas the effects of actinomycin-D and doxorubicin were greatly attenuated in these cells (Crawford et al., 1999). Furthermore, while caspase inhibitors blocked apoptosis induced by DNA-damaging agents in wild type MCF-7 cells, caspase inhibitors had little or no effect on apoptosis induced by sigma-2 ligands (Crawford et al., 1999). This suggests that sigma-2 receptors and DNA-damaging agents induce apoptosis via distinct mechanisms, with sigma-2 receptors apparently utilizing a caspase-independent pathway, as well as a pathway independent of p53 involvement.

The emergence of multidrug resistance has led to a search for drugs that sensitize resistant cancer cells to chemotherapeutic agents. A method of sensitizing drug resistant MCF-7/DOX human breast cancer cells to the antitumor compound doxorubicin using antipsychotic drugs such as phenothiazine analogs, pimozide and other agents is reported in U.S. Pat. No. 5,104,858. The reversal of multidrug resistance in WEHI 164 murine fibrosarcoma cells by certain lipophilic drugs administered with the antineoplastic compound actinomycin D is also reported (Hofsli et al., 1990). In addition, a synergistic killing effect between the sigma ligand BD737 and DTG, (+)-3-PPP, (+)-pentazocine, (+)-cyclazocine, benzomorphans and morphinans was reportedly observed in C6 glioma cells (Vilner et al., 1995).

However, there still remains a need for a more effective anticancer treatment method. In addition, there still remains a need for an anticancer treatment effective at killing both drug resistant and non-drug resistant cancer cells.

Therefore, it is an object of this invention to provide a more effective method of killing cancer cells. It is also an object of this invention to provide a method which kills both non-drug resistant and drug resistant cancer cells. It is further an object of this invention to provide a method to sensitize both non-drug resistant and drug resistant cancer cells to antitumor agents.

### SUMMARY OF THE INVENTION

The present invention concerns a method of sensitizing a cancer cell to an antitumor agent. In particular, the method concerns sensitizing a cancer cell to an antitumor agent comprising contacting the cancer cell with a subtoxic dose of a sigma-2 ligand and an effective amount of an antitumor agent. As used herein the term “cancer cell” means both a non-drug resistant and a drug resistant cancer cell.

The present invention relates to a sigma-2 agonist that can be used to potentiate the antineoplastic activity of a DNA-damaging agent. The DNA-damaging antineoplastic agents preferably do not have sigma binding activity. Non-limiting examples of such DNA-damaging agents are doxorubicin and actinomycin D. The sigma-2 agonist is used according to the invention at subtoxic concentrations that produce little or no cell killing when administered alone, yet still potentiate the antineoplastic activity of the DNA-damaging agent. Thus, the sigma-2 agonist of this invention exhibits a synergistic effect when combined with a DNA-damaging antineoplastic agent, as the total antineoplastic effect of the two is greater than the sum of the individual effects of the DNA-damaging antineoplastic agent and the subtoxic dose of a sigma-2 agonist. The ability of a sigma-2 agonist to potentiate antineoplastic compounds may allow for lower doses of the antineoplastic agent to be administered thereby maintaining efficacy but reducing life threatening toxicity.

An additional embodiment of the invention relates to the use of a sigma-2 agonist in combination with a DNA-damaging antineoplastic agent to treat tumors that are resistant to antineoplastic agents as a result of mutations in the p53 tumor suppressor gene. The antineoplastic effect of the sigma-2 agonist and DNA-damaging compound is also synergistic in drug resistant tumor cells.

An additional embodiment of the invention relates to the use of a sigma-2 agonist which is selective for the sigma-2 receptor. In one embodiment, the sigma-2 agonist has a greater binding affinity for the sigma-2 receptor relative to non-sigma receptors such as NMDA, AMPA and glutamate receptors and the like. In another embodiment, the sigma-2 agonist has a greater binding affinity for the sigma-2 receptor relative to the sigma-1 receptor.

### BRIEF DESCRIPTION OF THE DRAWINGS

FIG. 1. FIG. 1 shows a comparison of sensitivity to doxorubicin of MCF-7 cells and MCF-7/Adr⁻ cells. Cells were incubated in the presence of various concentration of doxorubicin. Cytotoxicity was determined by release of lactate dehydrogenase into culture media and expressed as percentage of total cell kill as described below. This figure is representative of a 48 hour time point. Each data point represents the mean and standard error of duplicate samplings from two culture wells at each concentration (4 samplings). Relative to MCF-7 cells, MCF-7/Adr⁻ cells were resistant to the cytotoxic effects of doxorubicin.

FIG. 2. FIG. 2 shows that sigma agonists and DNA-damaging antineoplastic agents induce apoptosis in various tumor cell lines. T47D cells (A, C, E, G, I) or MCF-7 cells (B, D, F, H, J) were incubated alone (A, B) or with the DNA-damaging agent, doxorubicin (C, D); the sigma receptor agonists CB-64D (E, F); haloperidol (G, H), or reduced haloperidol (I, J) for 48 hours. Cells were assayed for apoptosis by the TUNEL method, and photographed by fluorescence microscopy with a filter at 40× magnification. Green or yellowish-green cells are positive for DNA-fragmentation consistent with apoptosis. Red or orange-red cells are non-apoptotic propidium iodide staining nuclei. Compounds were used at a concentration of 100 μM.

FIGS. 3A and 3B. FIGS. 3A and 3B show the concentration-dependent effect of sigma-2 agonists on cell killing in different tumor cell lines. SKBr3 cells (FIG. 3A) and T47D (FIG. 3B) were incubated in the presence of various concentrations of the sigma-2 agonists CB-64D and CB-184. Cytotoxicity was determined by measuring the release of lactate dehydrogenase into culture media and expressed as percentage of total cell kill. Each data point represents the mean and standard error of duplicate sarnplings from two culture wells at each concentration (4 samplings). Each figure is representative of 3 or 4 experiments. Similar results were obtained using MCF-7 and MCF-7/Adr⁻ cell lines. CB-64D and CB-184 were cytotoxic in both cell lines. CB-184 was more potent than CB-64D.

FIGS. 4A and 4B. FIG. 4A shows various breast tumor cell lines and the status of their p53 gene. MCF-7 cells produce normal p53 protein, whereas p53 in MCF-7Adr⁻;, SKBr3, and T47D is defective. FIG. 4B shows the EC₅₀ in micromolar for cytotoxic response to sigma-2 agonist in breast tumor cell lines. The cytotoxic potencies of CB-64D and CB-184 was generally not affected by p53 status.

FIG. 5. FIG. 5 shows the effect of caspase inhibitors on sigma-2 agonists in the induction of apoptosis. MCF-7 cells were grown as described below and incubated with drugs in the presence or absence of the non-selective caspase inhibitor Z-VAD-FMK (50 μM) or the selective caspase-1 inhibitor Y-VAD-CHO (50 μM) for 48 hours. Apoptosis was determined using Annexin V binding. Treatments were as follows: control (no drug, A), Z-VAD-FMK (B), CB-64D 100 μM (C), doxorubicin 100 μM (D), CB-64D+Z-VAD-FMK (E); doxorubicin+Z-VAD-FMK (F), CB-64D+Y-VAD-CHO (G), doxorubicin+Y-VAD-CHO (H). Caspase inhibitors blocked apoptosis induced by doxorubicin but not by CB-64D.

FIGS. 6A, 6B and 6C. FIGS. 6A, 6B and 6C show the effect of caspase inhibitors on drug-induced cytotoxicity in MCF-7 cells. The results indicate that sigma-2 agonists and antineoplastic drugs have different mechanisms of action. MCF-7 cells were treated with various concentrations of CB-184, doxorubicin or actinomycin-D, either with or without DEVD-CHO (50 μM) or Z-VAD-FMK (50 μM) for the designated times. Cytotoxicity was determined by measuring the release of lactate dehydrogenase into culture media and expressed as a percentage of total cell kill. Each data point represents the mean and standard error of duplicate samplings from two culture wells for each treatment group (4 samplings). Each figure is representative of 3 experiments (* p<0.01, **p<0.001 by Student's two-tailed T-test). FIG. 6A shows the effect of DEVD-CHO on CB-184 cytotoxicity, FIG. 6B shows the effect of DEVD-CHO on actinomycin-D cytotoxicity, and FIG. 6C shows the effect of Z-VAD-FMK on doxorubicin cytotoxicity. Caspase inhibitors blocked cytotoxicity induced by actinomycin-D and doxorubicin, but not that induced by CB-184.

FIGS. 7A, 7B, 7C and 7D. FIGS. 7A, 7B, 7C and 7D show that combining antineoplastic drugs with a sigma agonist potentiates cytotoxicity. CB-184 (1 μM) was combined with doxorubicin (10 μM) in MCF-7 cells (24 hr, FIG. 7A; 48 hr, FIG. 7B), or with actinomycin-D in MCF-7/Adr⁻ (1 μg/ml at 24 hr, FIG. 7C; 0.1 μg/ml at 48 hr, FIG. 7D). Cytotoxicity was measured by lactate dehydrogenase release into culture media. Each bar represents the mean value and standard error of duplicate samplings from two culture wells for each treatment group (4 samplings). These figures are representative of 2-3 experiments (* p<0.001 comparing combined groups with each of the single drug treatment groups).

FIG. 8. FIG. 8 shows the effect of sigma agonists on doxorubicin-induced cytotoxicity in MCF-7/Adr⁻ tumor cells. MCF-7/Adr⁻ cells were incubated with various concentrations of doxorubicin alone or in the presence haloperidol (25 μM) or (±)-pentazocine (35 μM). Cytotoxicity was determined by measuring the release of lactate dehydrogenase into culture media and expressed as a percentage of total cell kill. Each data point represents the mean and standard error of duplicate samplings from two culture wells for each treatment group (4 samplings). This figure is representative of 3 experiments. Haloperidol and pentazocine potentiated the cytotoxicity of doxorubicin.

### DETAILED DESCRIPTION OF THE INVENTION

This invention involves the use of a sigma-2 agonist for treating patients suffering from neoplasia. Any ligand with sigma-2 agonistic activity can be used according to the method of the present invention. This is shown by the fact that the potentiating effect produced by sigma-2 agonism was observed in several different classes of sigma-2 agonists which indicates that the potentiating effect is found in all sigma-2 agonists.

A preferred sigma-2 agonist is one which exhibits a greater selectivity for the sigma-2 receptor relative to the sigma-1 receptor and, thus, is defined as sigma-2 selective agonist. More preferred is a sigma-2 selective agonist which exhibits about a 25-fold or greater selectivity for the sigma-2 receptor relative to the sigma-1 receptor. A more preferred sigma-2 selective agonist exhibits about a 50-fold or greater selectivity for the sigma-2 receptor relative to the sigma-1 receptor, and a most preferred sigma-2 selective agonist exhibits about a 100-fold or greater selectivity for the sigma-2 receptor relative to the sigma-1 receptor.

Non-limiting examples of such sigma-2 agonists include 5-substituted morphan-7-ones, 5,8-disubstituted morphan-7-ones, iboga alkaloids such as 12-methoxyibogamine (ibogaine) and 13-methoxyibogamine and the like (Bowen et al. 1995b), pentazocine and haloperidol. Preferred sigma-2 selective ligands include, but are not limited to, (+)-5,8-disubstituted morphan-7-ones and iboga alkaloids. Preferred (+)-5,8-disubstituted morphan-7-ones are CB-64D and CB-184.

The 5,8-disubstituted morphan-7-ones are illustrated by the following general formulae:

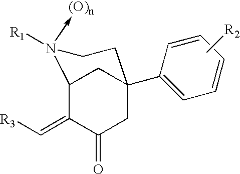

wherein:

n=0 or 1;

R₁=lower-alkyl, lower-alkenyl, cycloalkyl, lower-alkynyl, lower-alkylaryl, or hydrogen;

R₂=lower-alkyl, lower-alkoxy, lower-alkylamino, hydroxy, amino, nitro, halo, azido, or hydrogen

R₃=aryl, alkylaryl, lower-alkyl, cycloalkyl, lower-alkenyl, lower-alkynyl, lower-alkylaryl, lower-alkoxy, lower-alkylamino, hydrogen, or hydroxy.

The term “lower-alkyl” as used herein means linear or branched hydrocarbon chains having from one to about ten carbon atoms and thus includes methyl, ethyl, propyl, isopropyl, n-butyl, sec-butyl, and the like.

The term “lower-alkenyl” means branched or unbranched unsaturated hydrocarbon radicals of from two to about ten carbon atoms and thus includes 1-ethenyl, 1-propenyl, 2-propenyl, 1-methyl-2-propenyl, isopropenyl, 2-butenyl, isobutenyl, and the like.

The term “cycloalkyl” means bridged or unbridged hydrocarbon ring systems having from three to about ten carbon atoms and thus includes cyclopropyl, cyclobutyl, cyclopentyl, cyclohexyl, cycloheptyl, cyclooctyl, cyclodecyl, norbornyl, adamantyl, and the like.

The term “lower-alkynyl” as used herein means branched or unbranched unsaturated hydrocarbon radicals of from two to about ten carbon atoms and thus includes 1-ethynyl, 1-propynyl, 2-propynyl, 2-butynyl, 1-methyl-2-propynyl, and the like.

The term “alkaryl” as used herein means a substituted or non-substituted aryl wherein the substitution can be hydrogen or one to about 10 carbon atoms.

The term “lower-alkoxy” as used herein means linear or branched alkyloxy substituents having from one to about ten carbon atoms and thus includes methoxy, ethoxy, propoxy, isopropoxy, butoxy, sec-butoxy, and the like.

The term “lower-alkylamino” as used herein means linear or branched alkylamino substituents having from one to about ten carbon atoms and thus includes methamino, ethylamino, propylamino, isopropylamino, butylamino, sec-butylamino, and the like.

The terms “halogen,” “halide,” or “halo” as used herein mean bromine, chlorine, iodine or fluorine.

The term “aryl” as used herein means optionally substituted phenyl, optionally substituted naphthyl, optionally substituted thienyl, optionally substituted furyl, optionally substituted imidizoyl, optionally substituted pyridyl, optionally substituted thienyl, indolyl, optionally substituted quinolyl, and the like.

It will be appreciated that the sigma-2 agonist can possess one or more asymmetric carbon atoms and is thus capable of existing in a number of stereoisomeric forms, i.e. enantiomers and diastereomers. Unless otherwise specified herein, the invention is intended to extend to each of these stereoisomeric forms and to mixtures thereof, including the racemates. In some cases there may be advantages, i.e. greater potency, to using a particular enantiomer when compared to the other enantiomer or the racemate in the methods of the instant invention and such advantages can be readily determined by those skilled in the art. The separate enantiomers may be synthesized from chiral starting materials or the racemates may be resolved by conventional procedures which are well known in the art of chemistry such as chiral chromatography, fractional crystallization of diastereomeric salts and the like. Likewise, the diastereomers can be separated by conventional procedures which are well known in the art of chemistry such as chromatography, fractional crystallization and the like.

The synthesis of the morphanones is known to those of skill in the art. For example, several groups (Bertha, 1994; Bertha, 1995 and Bowen, 1995) describe the preparation of a number of morphanones within this class of compounds as well as the structure activity relationships of these compounds. In vitro binding assays of various stereoisomers were performed to study binding to sigma-1 and sigma-2 receptor subtypes. It was found that the dextro isomers of the morphanones had a high degree of selectivity for the sigma-2 receptor subtype.

When designing and synthesizing a sigma-2 agonist for use according to the present invention, a consideration of the lipophilicity and basicity of the sigma-2 agonist should be made. Evidence indicates that sigma ligands must cross the cell membrane and interact with sigma-2 receptors located in subcellular organelles in order to produce the effects on calcium and cell viability described herein. For example, human SK-N-SH neuroblastoma cells were used to measure rapid changes in Ca⁺⁺ concentration or cytotoxicity after a 24 hr treatment (LDH release). In both assays, the potency of a series of sigma-2 agonists increased as the lipophilicity (Log P) values increased. Furthermore, the activity of the compounds was affected by the pH of the media. For compounds which are protonated at physiological pH, raising the medium pH leads to increased deprotonation, and thus increases lipophilicity, whereas lowering the pH decreases the lipophilicity. In addition, the antagonists BD1047 and BD1063, were ineffective in the calcium assay at pH 7.2, but effectively attenuated agonist activity at pH 8.2 (Bowen et al., 1999). These data indicate that at pH 8.2, both agonist and antagonist are accessible to the receptor, whereas at pH 7.2 the very hydrophilic antagonists are largely excluded from access to the receptor while the more lipophilic agonists have some access. Cytotoxicity ED₅₀ values, determined at pH 7.2 and 8.2, differed by as much as 20-fold and were always lower at pH 8.2. Correcting the ED₅₀ values at each pH for the fraction of deprotonated ligand present led to normalization to a much lower value which was similar at each pH (Bowen et al., 1999). However, these results were not due to effects of pH on ligand binding. Raising pH from pH 7.2 to 8.2 had no effect on the affinity of agonists or antagonists for the sigma-2 receptor or on the apparent receptor number, an increase in either of which might result in an increase in agonist potency. These data indicate that sigma-2 agonists which are deprotonated and/or more lipophilic will potentially perform better in vivo as the ligands must cross the membrane and act on intracellular sigma-2 receptors. Therefore, sigma-2 agonist which are more lipophilic and have fewer sites available for protonation are preferred for use according to this invention.

The MCF-7/Adr⁻ cells with mutant p53 have been shown to over-express the MDR gene product, p-glycoprotein (Ogretmen, 1997). P-glycoprotein enhances the efflux of hydrophobic compounds that are often toxic to cells. Doxorubicin is a substrate for MDR and over-expression of MDR results in phenotypic resistance to doxorubicin, even in the presence of wild-type p53 (Thor et al., 1998). Sigma-2 agonists have been shown to reduce the expression of the MDR gene (Bowen et al., 1997). However, this is not likely to be the predominant mechanism for the potentiation observed, since it occurs in MCF-7 cells that are sensitive to doxorubicin, and do not over-express MDR.

The present invention comprises a method for partially or totally inhibiting formation of, or otherwise treating (e.g., reversing or inhibiting the further development of) tumors in a mammal, for example, a human, by administering to the mammal a subtoxic amount of a sigma-2 agonist or a subtoxic combination of sigma-2 selective agonists in conjunction with an antineoplastic agent. The subtoxic dose of the sigma-2 selective agonist(s) and effective dose of antineoplastic agent can be administered alone or in conjunction with other drugs, such as other anti-cancer drugs or in a pharmaceutical composition further comprising an acceptable carrier or diluent, and, optionally, other drugs. Administration can be by any means which are conventional for pharmaceutical, preferably oncological, agents, including oral and parenteral means such as subcutaneously, intravenously, intramuscularly and intraperitoneally, nasally or rectally.

The method of the present invention provides for a higher therapeutic index of safety compared to current methods of cancer treatment. This is because the DNA-damaging antineoplastic agent can be administered to a patient in lower doses when given in conjunction with a sigma-2 agonist. This synergistic combination is effectively targeted at cancer cells because tumor tissues express a much higher density of sigma receptors than normal tissues (Bern et al., 1991). For example, in a pilot study on breast tumor tissue, a very high density of sigma receptors was found in the tumor biopsy tissue while little or no sigma binding was detected in samples from surrounding normal tissue (John et al., 1996). Furthermore, though tumor cells already express a higher density of receptors than normal cells, the density of sigma-2 receptors increases even higher in more rapidly proliferating cells (Mach et al., 1997). This higher receptor number may increase the sensitivity of tumor cells to the apoptotic effect of a sigma agonist and the combination of a sigma agonist with a DNA-damaging antineoplastic agent, allowing the use of doses which will have less of an affect on normal cells. Thus, the synergistic antineoplastic effect of the combination of sigma-2 agonist and DNA-damaging antineoplastic agent will have a greater effect on cancer cells due to the relatively high level of sigma-2 receptor expression in cancer cells.

The dosage of the sigma-2 selective agent to be administered to the mammal, such as a human, is a subtoxic amount. Preferably, the subtoxic amount is an amount that produces little or no killing of cancer cells. More preferably, the subtoxic amount is an amount that produces little or no effect on cancer cell morphology. The sigma-2 selective agonist can be administered in a single dose or multiple doses in a given period of time (e.g., a single daily dose or two or more doses a day). The subtoxic dose depends on the type of cancer being treated. Neuroblastoma cells are sensitive at lower concentrations of sigma-2 agonist than breast cells are. Toxicity also has a temporal component where lower doses may require more time before toxicity is evident. In the LDH assay, toxicity is generally not seen in breast cell lines below 1 μM CB-184 at 24 hrs and mildly increased LDH release may be observed at 48 hrs compared to controls. CB-184 is the most potent sigma-2 selective agent in intact cells. CB-64D, another sigma-2 selective agent is about 10 fold less potent than CB-184.

Antineoplastic agents for use according to this invention are those antineoplastic agents which selectively damage the DNA of cancer cells. Non-limiting examples of such antineoplastic agents are doxorubicin and actinomycin. Antineoplastic agents encapsulated in liposomes are preferred. For example, doxorubicin encapsulated in liposomes generally targets tumors more efficiently and produces less systemic toxicity allowing lower doses to be given.

A therapeutically effective amount of an antineoplastic agent is an amount when given in conjunction with a subtoxic dose of a sigma-2 selective agonist which is sufficient to inhibit (partially or totally) formation of a tumor or other malignancy or to reverse development of a solid tumor or other malignancy or prevent or reduce its further progression. For a particular condition or method of treatment, the dosage is determined empirically, using known methods, and will depend upon factors such as the biological activity of the particular compound employed; the means of administration; the age, health and body weight of the recipient; the nature and extent of the symptoms; the frequency of treatment; the administration of other therapies; and the effect desired. Doses of anti-neoplastic agents vary tremendously depending on the agent and they are frequently dosed according to body surface area rather than body weight. For doxorubicin, dose may range from 60-75 mg/m² every 3 weeks, or 20 mg/m² per week; or 40-60 mg/m² every three weeks if combinations of agents are used. Actinomycin D is dosed 400-600 μg/m² for 5 days or 35-50 μg/m² by perfusion.

Further, the sigma-2 agonists and antineoplastic agents can be administered by regional isolation perfusion. This is where an organ or limb is perfused with sigma-2 agonists and antineoplastic agents in lieu of, or in addition to, systemic administration. In addition, the present invention provides for the co-administration of more than one sigma-2 agonist and/or more than one antineoplastic agent during the course of treatment.

The compounds of the present invention can be administered in conventional solid or liquid pharmaceutical administration forms, for example, uncoated or (film-)coated tablets, capsules, powders, granules, suppositories or solutions. These are produced in a conventional manner. The active substances can, for this purpose, be processed with conventional pharmaceutical aids such as tablet binders, fillers, preservatives, tablet disintegrants, flow regulators, plasticizers, wetting agents, dispersants, emulsifiers, solvents, sustained release compositions, antioxidants and/or propellant gases. The administration forms obtained in this way typically contain from about 1 to about 90% by weight of the active substance.

The pharmaceutically acceptable carrier and compounds of this invention are formulated into unit dosage forms for administration to a patient. The dosage levels of active ingredient (i.e. compounds of this invention) in the unit dosage may be varied so as to obtain an amount of active ingredient effective to achieve antineoplastic activity in accordance with the desired method of administration. The selected dosage level therefore depends upon the nature of the active compound administered, the route of administration, the desired duration of treatment, and other factors. If desired, the unit dosage may be such that the daily requirement for active compound is in one dose, or divided among multiple doses for administration, e.g., two to four times per day.

Sigma-2 agonists are useful for a wide variety of cancer chemotherapy. They can be used either alone or in combination with other antineoplastic agents. For example, experiments showed that treatment of “wild type” MCF-7 breast tumor cells with a concentration of sigma-2 agonist which produced little or no apparent effect on cell morphology or viability resulted in potentiation of the cytotoxic effect of actinomycin-D and doxorubicin. In addition, previous experiments have shown that a 24 hour exposure of human SK-N-SH neuroblastoma or rat C6 glioma cells to a sub-toxic dose of sigma-2 agonists caused a marked decrease in the expression of mRNA for p-glycoprotein, a drug efflux pump believed to play a major role in the development of drug resistance in cancer cells (Bowen et al., 1997). Taken together, these results show that sigma-2 receptors are capable of mediating a reversal of drug resistance in tumor cells. Thus, sigma-2 agonists are useful alone as antineoplastic agents at doses which induce apoptosis or could be used at sub-toxic doses in combination with common antineoplastic agents to reverse drug resistance, allowing for the use of lower doses of the agent and thereby reducing adverse effects.

The evaluation of LDH release as a method to quantify cell death does not necessarily distinguish apoptotic from necrotic cell death. Therefore, cell death was also monitored by both the TUNEL assay and through Annexin V binding. At the appropriate sigma ligand concentration and duration, up to 100% cell killing can be observed with the potent and selective agents CB64D and CB184 when analyzed by LDH release, the TUNEL assay or Annexin V binding, though the optimal duration may vary between the assays. These observations establish that the mode of cell death is apoptotic.

Caspases are a family of cysteine-aspartyl proteases that are the executioners of apoptotic signals from diverse stimuli including receptor activation (eg. Fas ligand, TNFα), DNA-damaging agents (e.g., doxorubicin, alkylating agents), hypoxia, growth factor deprivation or ionizing radiation. The targets of caspases include a vast array of cytoskeletal proteins, cell cycle regulatory proteins and nuclear matrix proteins such that the proteolytic cleavage of these targets is consistent with the morphological and biochemical alterations characteristic of apoptosis. Most apoptotic signals initiate a cascade of sequential caspase activation (Cohen, 1997). However, caspase independent pathways have also been described. In order to further characterize the apoptotic effect observed here, the effects of the caspase inhibitors on the actions of sigma-2 agonists were compared with doxorubicin and actinomycin D. Surprisingly, inhibitors of caspases had no effect on cytotoxicity of the sigma-2 agonists, whereas the response to the anti-neoplastic agent was prevented. Further, phosphatidylserine externalization, a feature of apoptosis (Fadock et al., 1992), is not blocked by caspase inhibitors in cells treated with sigma-2 agonists.

Sigma-2 agonists are equipotent in killing cells with mutant and wild-type p53 genes and can potentiate anti-neoplastic drug effects in cancer cells such as breast tumor cells. This has tremendous implications for clinical practice. Unfortunately, the utility of toxic doses of potent sigma-2 receptor ligands for treating tumors may be limited since many tissues normally express high-densities of the receptor and chronic exposure to these agents may have deleterious effects. However, the ability of these compounds to potentiate anti-neoplastics means that lower doses of the sigma compounds are required to enhance the effects of drugs which already have some limited selectivity for tumors. Furthermore, sigma-2 agonists are able to potentiate at a dose which alone is not cytotoxic. This phenomenon may also result in reversal of drug resistance in tumors at concentrations of the drug that reduce the adverse side effects.

Some sigma-2 agonists are clinically available and also display the potentiation phenomena. These include, but are not limited to the butyrophenones, droperidol, haloperidol, and racemic pentazocine (Talwin). Therefore, these agents which have other actions useful in cancer patients can be used to potentiate anti-neoplastic activity. For example, haloperidol has been used to treat emesis and has been administered for psychiatric disorders in oral doses of up to 100 mg, 20-400 mg intramuscularly which typically produce plasma concentrations of between about 4 to about 20 ng/ml. Pentazocine has been administered to treat moderate to severe pain in doses of up to 100 mg orally every four hours, 30 mg IV and 60 mg IM. These doses typically produce peak plasma concentrations of about 160 ng/ml. The doses of haloperidol and pentazocine that potentiate doxorubicin and actinomycin D are higher than the standard doses given to treat emesis and pain during chemotherapy. In an embodiment of the invention, haloperidol, pimozide or pentazocine are administered in an amount greater than about 1 mg parenterally or orally per day. Preferably, haloperidol, pimozide or pentazocine are administered in an amount between about 5 mg to about 400 mg parenterally or orally per day. Alternatively, haloperidol, pimozide or pentazocine are administered in an amount greater than about 10 mg, about 25 mg or about 40 mg parenterally or orally per day with an upper limit of about 500 mg parenterally or orally per day. In a preferred embodiment of the invention, haloperidol, pimozide or pentazocine are administered orally to a patient 2 or 3 days prior to chemotherapy to attain a steady state blood level of haloperidol, pimozide or pentazocine. Thus, these drugs may be co-administered with an antineoplastic agent for a limited duration to limit toxicity by allowing for lower dosing of the antineoplastic agent.

Sigma-2 receptor agonists induce apoptosis in various tumor cells in a manner apparently independent of both p53 and caspase activation. Further, sigma-2 agonists potentiate the action of DNA-damaging agents at doses which are not cytotoxic. This suggests that sigma-2 receptors utilize an apoptotic pathway distinct from that utilized by DNA-damaging agents and other apoptotic stimuli. Thus, sigma-2 receptors represent a novel therapy for use with antineoplastic agents to control cancer.

All publications, patents and articles referred to herein are expressly incorporated herein in toto by reference thereto. The following examples are presented to illustrate the present invention but are in no way to be construed as limitations on the scope of the invention. It will be recognized by those skilled in the art that numerous changes and substitutions may be made without departing from the spirit and purview of the invention.

### EXAMPLES

### Methods

Cell Culture

Human breast tumor cell lines (MCF-7, T47D, SKBr3, and MCF-7/Adr⁻) were cultured in DMEM containing 3.7 g/liter Na₂HCO₃, fetal bovine serum (10%), and insulin (10 mg/liter). For cytotoxicity assays, cells were transferred to DMEM+Ham's nutrient mixture F-12 (without phenol red), with 1.2 g/liter Na₂HCO₃. Cells were seeded at 100,000 cells/well. Human SK-N-SH neuroblastoma and DU-145 prostate carcinoma cells were cultured in DMEM, supplemented with fetal bovine serum (10%).

Cytotoxicity Assay

Cell death was assessed by release of lactate dehydrogenase (LDH) into the culture medium using the CytoTox 96 kit from Promega (Madison, Wis.). The method used was as specified by manufacturer with minor modifications. Cells were plated and cultured in 24-well plates for 1 to 2 days prior to the experiment. Cells were treated in the plates under the specified drug conditions. Following treatment, the medium was transferred to centrifuge tubes and centrifuged to remove cell debris. Supernatant (50 μl) was transferred to a 96-well plate, to which was added substrate mix in assay buffer, followed by stop solution after a 30 min incubation. Formation of formazan was monitored at 490 nm in a plate reader. Values are expressed relative to total lysis controls (Triton X-100 was used to determine value for 100% cell kill) and no drug-controls. Doxorubicin and caspase inhibitors (Z-VAD-FMK, Y-VAD-CHO, DEVD-CHO) were obtained from Calbiochem (San Diego, Calif.) and actinomycin-D from Sigma (St. Louis, Mo.).

Detection of Apoptosis by Annexin V binding

The early stages of apoptosis are characterized by translocation of phosphatidyl serine (PS) from the inner surface of the plasma membrane to the outer surface of the membrane. Externalized PS can then be detected using Annexin V, a protein with high affinity for PS. This was carried out using the ApoAlert Annexin V Apoptosis Kit (Clontech, Palo Alto, Calif.) according to the manufacturer's specifications. After incubation with sigma ligands at the concentrations and times specified, live cells (without fixing) were incubated with annexin V-FITC and propidium iodide, and were investigated using fluorescence microscopy. Green staining shows externalized PS, while orange color revealed damage of cell membrane and penetration of propidium iodide to the cell interior, which occurs at the later stages of the process. Filters used: excitation 450 nm, barrier 535 nm.

Detection of Apoptosis by the TUNEL Method

DNA fragmentation occurring during apoptosis can be detected by incorporating fluorescein-12-dUTP at the 3′-OH DNA ends using the enzyme, terminal deoxynucleotidyl transferase (TdT). TUNEL (TdT-mediated dUTP Nick-End Labeling) was performed using the Apoptosis Detection System, Fluorescein Kit (Promega, Madison, Wis.) according to the manufacturer's specification. After treatment with sigma ligands at the concentrations and times specified, cells were fixed with 4% formaldehyde, permeabilized with Triton X-100, and incubated with fluorescein-12-dUTP and terminal deoxynucleotidyl transferase and then with propidium iodide. Propidium iodide stains the DNA in both apoptotic and non-apoptotic cells with orange-red color. Fluorescein produces yellow-green fluorescence, indicating fragmented DNA within the nucleus of apoptotic cells. Filters used: excitation 450-490 nm, barrier 520 nm.

Sigma Receptor Ligands

Haloperidol and reduced haloperidol are subtype non-selective sigma ligands (Bowen, 1990) and were obtained from Research Biochemicals Inc. (Natick, Mass.). Racemic pentazocine was obtained from the National Institute of Drug Abuse. The 5-phenylmorphans CB-64D and CB-184 are sigma-2 subtype-selective agonists synthesized as described in the literature (Bowen, 1995a; Bertha et al., 1995). CB-64D: (+)-1R,5R-E-8-benzylidene-5-(3-hydroxyphenyl)-2-methylmorphan-7-one; CB-184: (+)-1R,5R-E-8-(3,4-dichlorobenzylidene)-5-(3-hydroxyphenyl)-2-methylmorphan-7-one

### Example 1

### Induction of Apoptosis in Various Tumor Cell Lines by Sigma Ligands

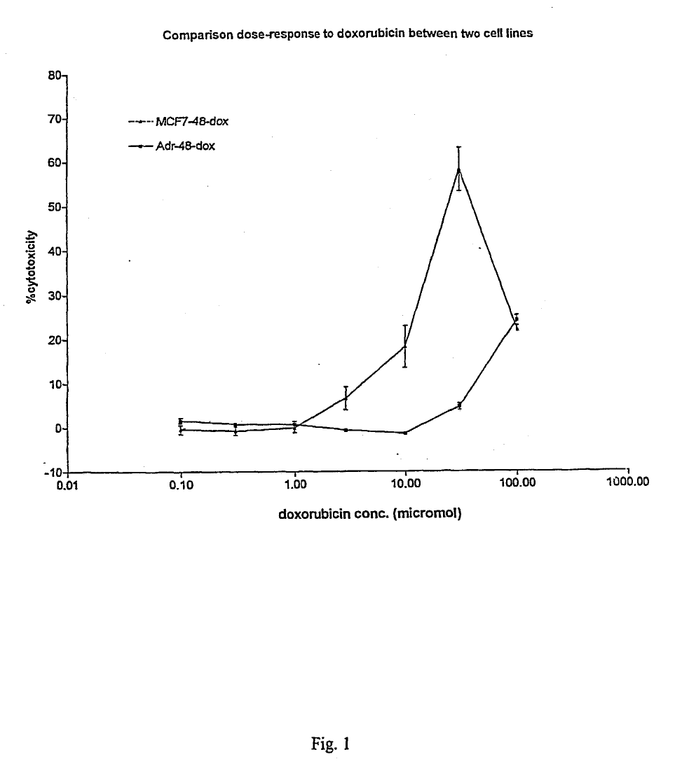

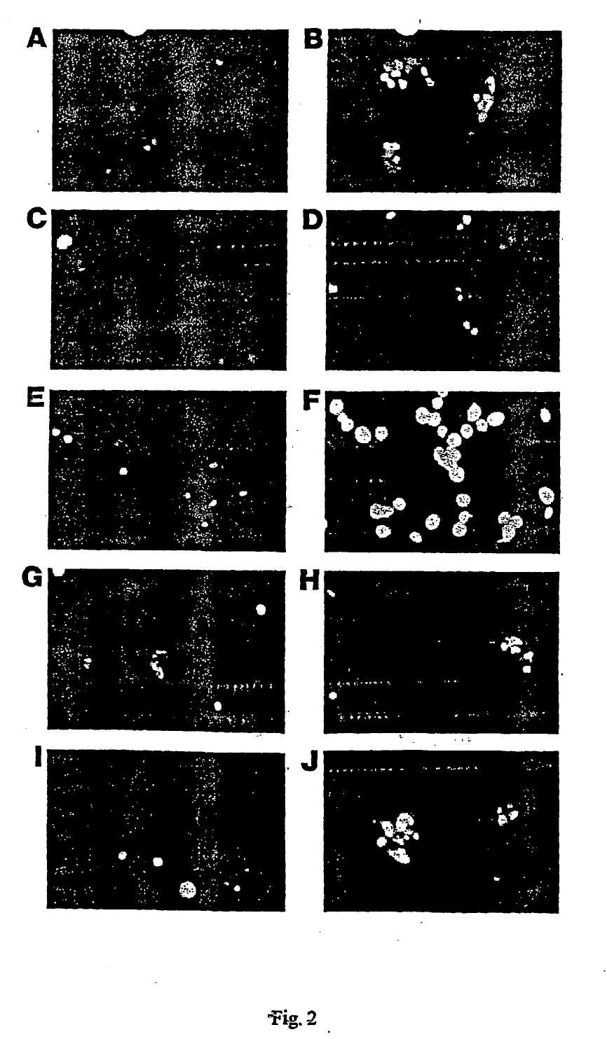

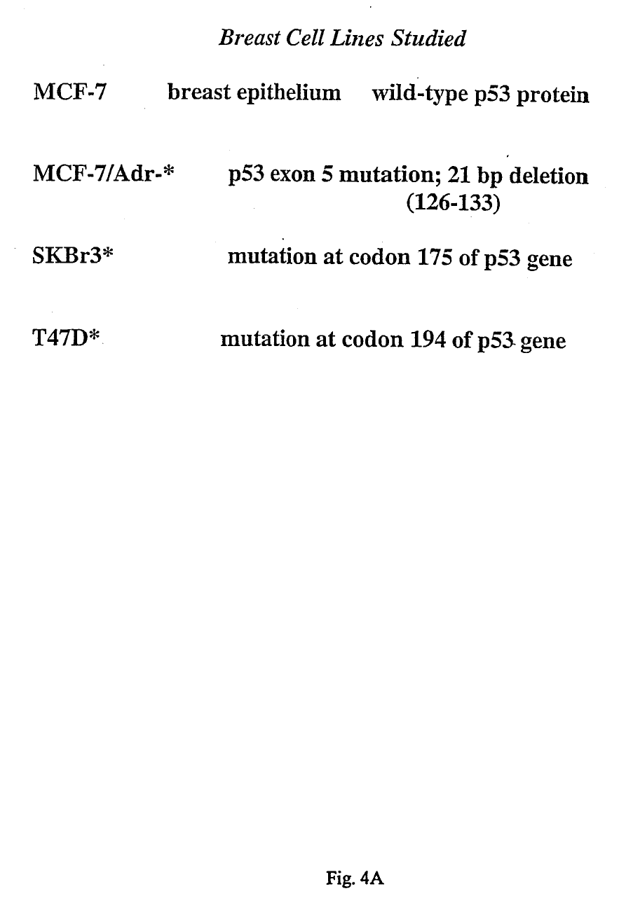

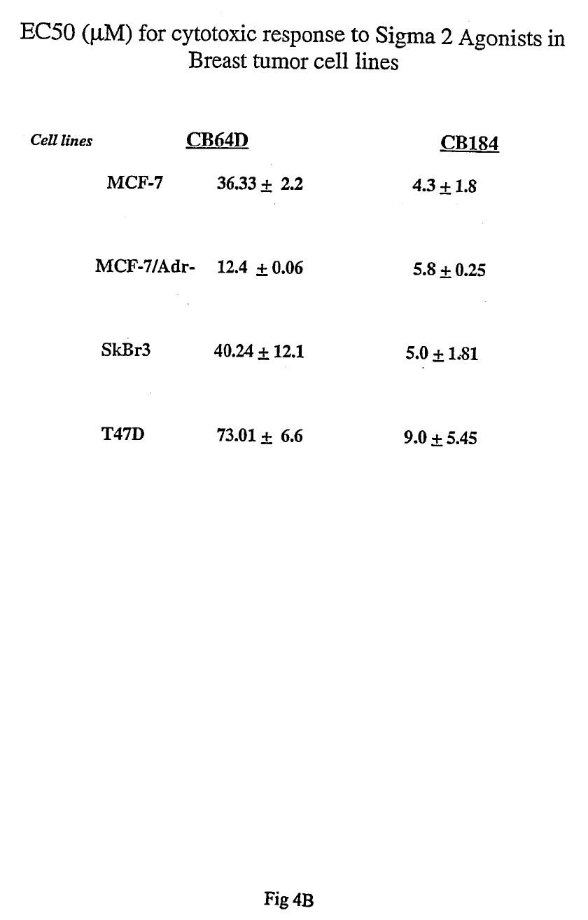

The cell lines examined in this study are presented in FIG. 4A along with the p53 genotype. The presence of p53 mutations renders some of these strains resistant to certain anti-neoplastic agents. FIG. 1 shows that the MCF-7/Adr⁻ displays diminished sensitivity to adriamycin compared to MCF-7 cells with WT p53. Various lines of cultured cells were incubated with sigma ligands and anti-neoplastic agents and the cells were assayed by the TUNEL method to assess drug-induced effects and potential mechanisms. Sigma ligands including CB64D, haloperidol, reduced haloperidol and antineoplastic agents such as doxorubicin, produced positive results in the TUNEL assay in MCF-7 and T47D cells (FIG. 2). The sigma-2 subtype selective ligand, CB64D, at a concentration of 100 μM produced extensive apoptotic changes in MCF-7 by 48 hours. In some experiments, up to 100% of cells visualized displayed apoptotic nuclei. The subtype non-selective sigma ligands, haloperidol and reduced haloperidol produced apoptotic nuclei, but less extensively than the more potent CB64D. Similarly, apoptosis was induced by doxorubicin and other antineoplastic drugs (actinomycin D, cyclophosphamide) in T47D and SKBr3 cell lines.

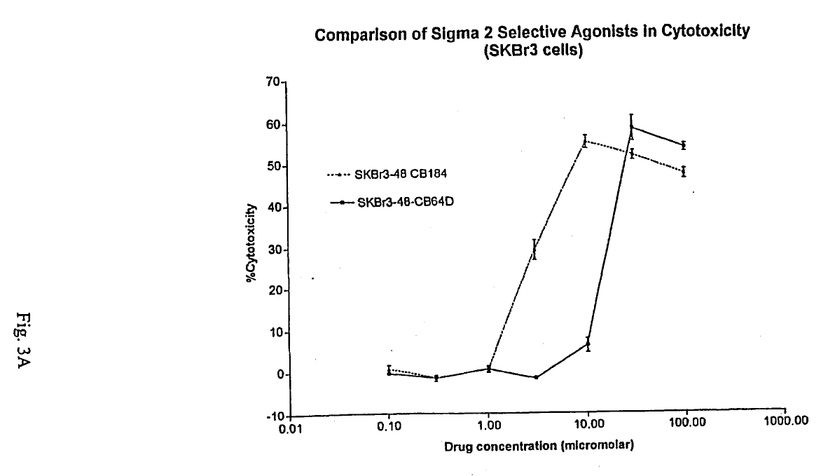

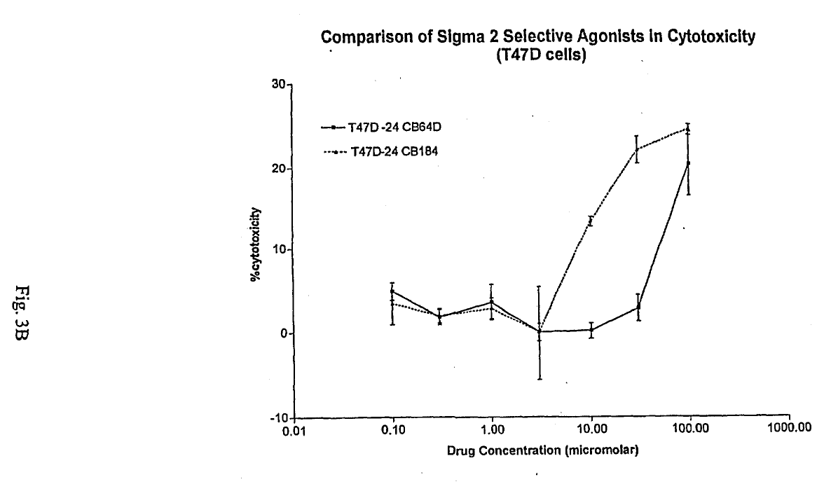

To quantify cell death induced by these agents, cultured cells were incubated in the presence of various concentrations of sigma-2 selective ligands, CB64D and CB184, at different time intervals, and cytotoxicity quantified by the release of lactate dehydrogenase (LDH) into cell culture media. A concentration dependent effect of the compounds on cytotoxicity in two cell lines, SkBr3 and T47D is shown in FIGS. 3A and 3B, respectively. The potency of these compounds in inducing cell death is compared for four different cell lines in FIG. 4B. CB184 exhibits the greatest potency in all cell lines tested. The cytotoxicity of CB-64D and CB-184 in drug-resistant cell lines with p53 mutations (MCF-7/Adr⁻, SKBr3, and T47D) was comparable to that in the non-drug resistant MCF-7 cells which have “wild-type” p53.

### Example 2

### Involvement of Caspases in Cell Death

Caspases (cysteine aspartyl proteases) play a central role in the pathway of apoptosis by executing the apoptotic signals (Cohen, 1997). Both selective and non-selective inhibitors of caspases have been developed as biochemical tools to help dissect the pathways by which an apoptotic signal is transmitted. The ability of different caspase inhibitors to abrogate apoptosis induced by sigma ligands and some anti-neoplastic drugs was compared.

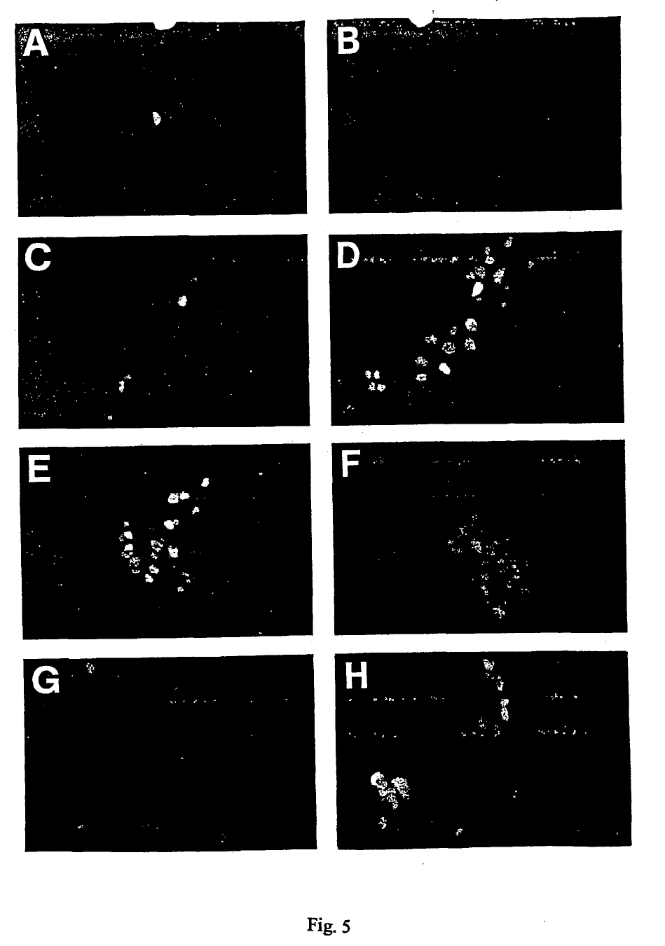

MCF-7 cells were treated with the sigma ligand CB64D (100 μM) in the presence or absence of YVAD-CHO (50 μM), an inhibitor of caspase 1 or ZVAD-FMK (50 μM) an inhibitor of all known caspases, for various time periods. Apoptosis was determined by the binding of Annexin V to the treated cells. The apoptotic changes occurring in the CB-64D treated cells were unaffected by co-treatment with caspase inhibitors (FIG. 5).

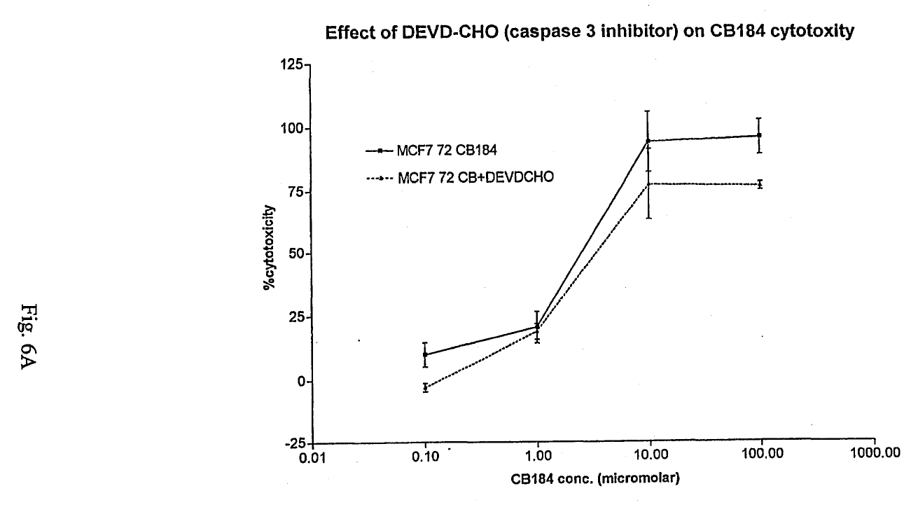

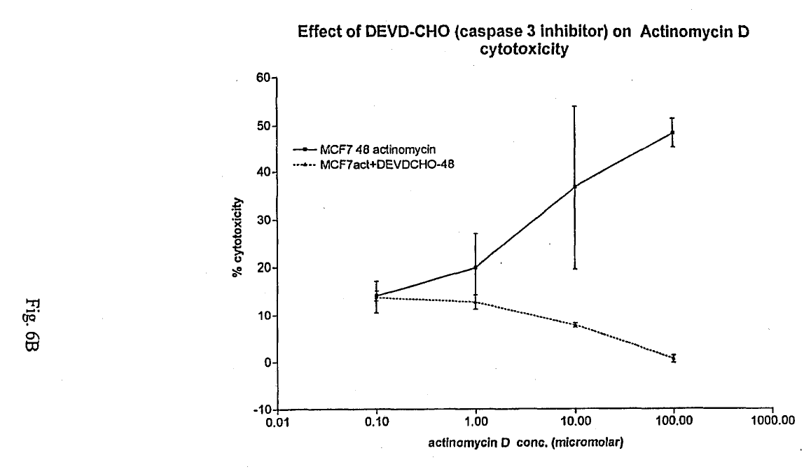

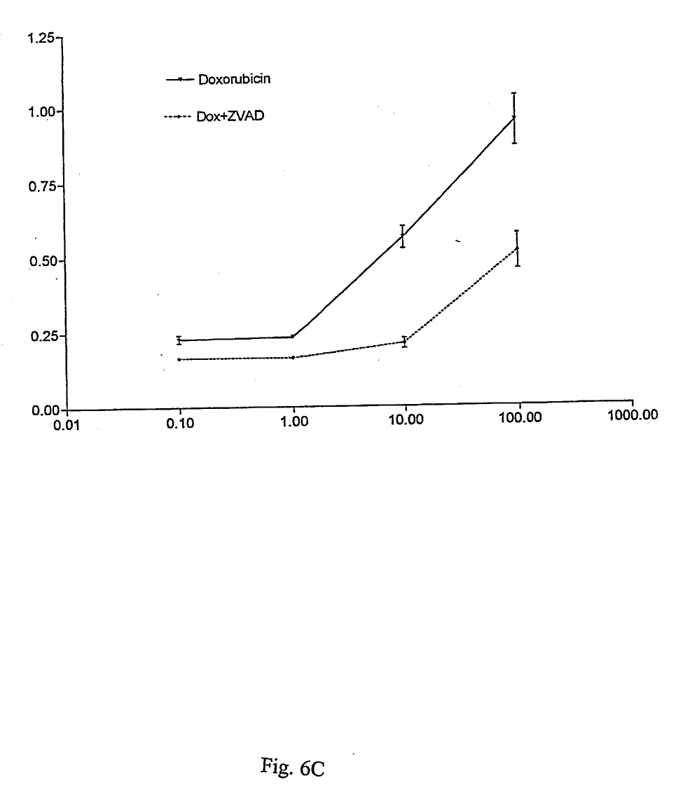

Actinomycin D has been shown to produce apoptosis by either inhibition of nucleic acid synthesis or by intercalation with DNA base pairs leading to the induction of p53 (Martin et al., 1990). MCF-7 cells were treated with varying concentrations of either CB184 or actinomycin D in the presence or absence of DEVD-CHO (50 μM), a specific inhibitor of caspase 3. Cytotoxicity was assessed by LDH release. DEVD-CHO abrogated actinomycin D cytotoxicity at its higher doses (FIG. 6B) whereas in cells treated with CB 184, there was no difference in the presence or absence of DEVD-CHO on cell killing (FIG. 6A). These observations were consistent with the effects of caspase-inhibitors in the presence of the sigma-2 selective CB64D in the Annexin V binding experiments above. Doxorubicin induced a dose-dependent increase in LDH-release, where the effect of the lower doses was abolished by Z-VAD-FMK (FIG. 6C). These results indicate that sigma-2 agonists induce apoptosis by a caspase-independent mechanism.

### Example 3

### Sigma Agonists Potentiate Antineoplastic Agents

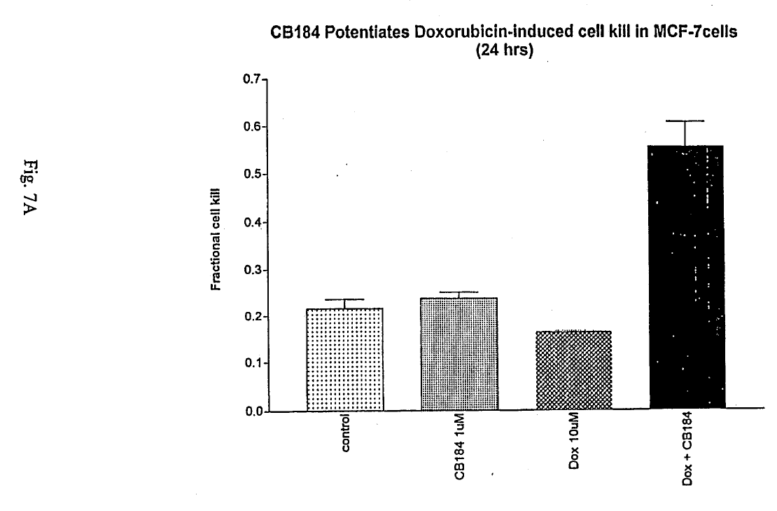

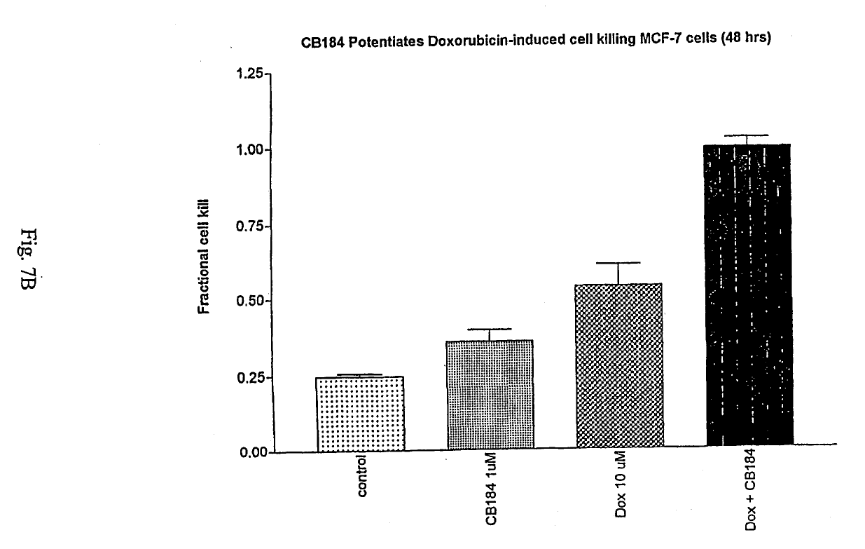

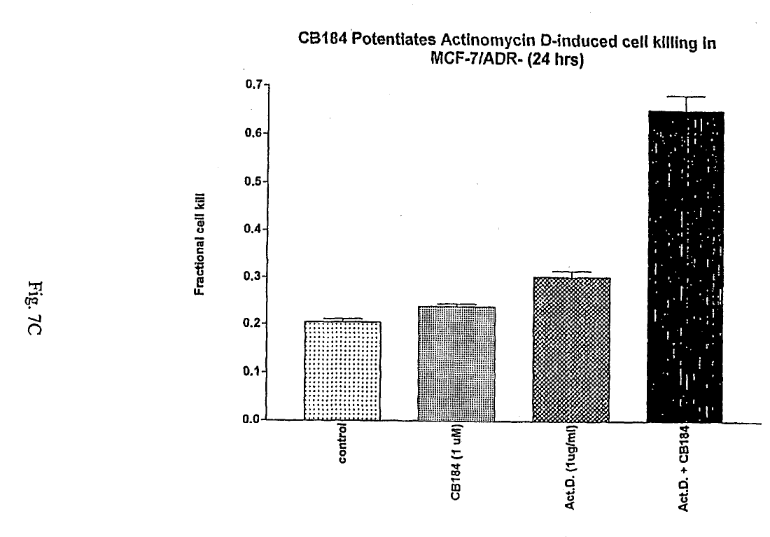

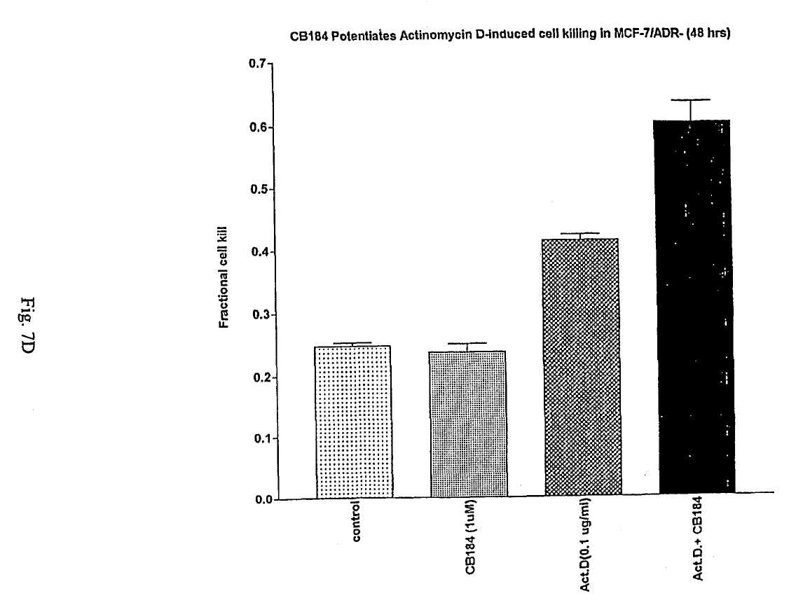

The combination of the sigma-2 agonist with anti-neoplasic agents, at concentrations of the individual agents producing modest to no effects on cell killing, resulted in substantial cytotoxicity. Clear synergistic effects were observed at 24 and 48 hours when CB184 (1 μM) was combined with 10 μM of doxorubicin (FIGS. 7A and 7B). This effect was observed in MCF-7 cells and also in MCF-7/Adr⁻, but less pronounced. Cytotoxicity was also potentiated in MCF-7/Adr⁻ cells with the combination of CB184 and actinomycin D (1 μg/ml at 24 hours or 0.1 μg/ml at 48 hours; FIGS. 7C and 7D).

### Example 4

### Effect of Sigma Agonists on Doxorubicin-Induced Cytotoxicity in MCF-7/Adr⁻ Tumor Cells

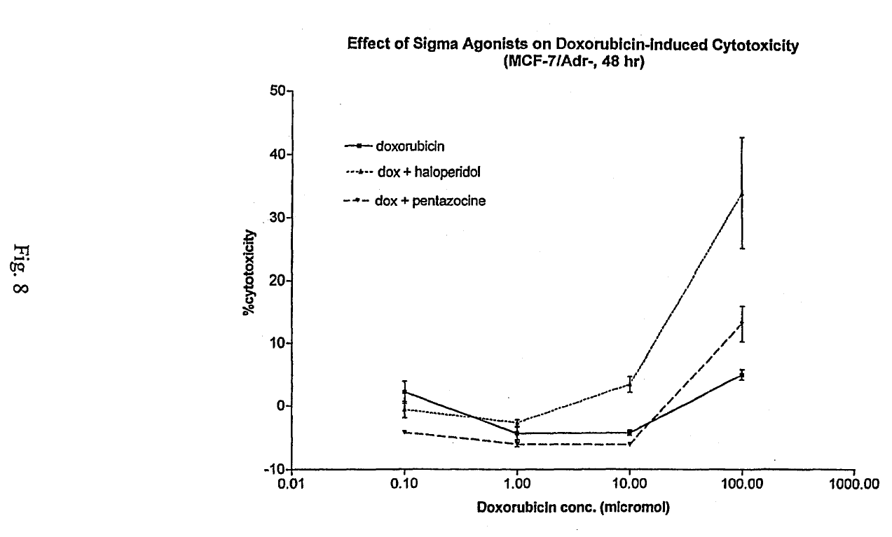

To determine if clinically available sigma agonists displayed similar effects as CB184, the non-selective sigma agonists haloperidol and pentazocine (a racemic mixture) were combined with doxorubicin at various concentrations. MCF-7/Adr⁻ cells were incubated with various concentrations of doxorubicin alone or in the presence haloperidol (25 μM) or pentazocine (35 μM). Cytotoxicity was determined by measuring the release of lactate dehydrogenase into culture media and is expressed as a percentage of total cell kill. The results are shown in FIG. 8. In MCF-7/Adr⁻ cells, haloperidol (25 μM) markedly potentiated doxorubicin, and to a lesser extent pentazocine (35 μM) potentiated the higher doses of doxorubicin. Little or no potentiation was observed by these agents when combined with doxorubicin in MCF-7 cells.

This data demonstrates that other agents from distinct pharmacologic classes that share sigma-2 agonist activity can potentiate DNA-damaging antineoplastic agents such as doxorubicin in a drug-resistant cell line. The pentazocine used in this experiment is a racemic mixture. (−)-Pentazocine has sigma-2 affinity whereas (+)-pentazocine binds very weakly.

### REFERENCES

Bem et al. (1991) Overexpression of sigma receptors in non-neural human tumors, Cancer Res. 51:6558-6562.

Bertha et al. (1994) A Marked Change of Receptor Affinity of the 2-Methyl-5-(3-hydroxyphenyl) morphans upon Attachment of an (E)-8-Benzylidene Moiety: Synthesis and Evaluation of a New Class of □ Receptor Ligands, J. Med. Chem. 37:3163-3170.

Bertha et al. (1995) (E)-8-Benzylidene Derivatives of 2-Methyl-5-(3-hydroxyphenyl) morphans: Highly Selective Ligands for the □₂ Receptor Subtype, J. Med. Chem. 38:4776-4785.

Bowen et al. (1999) Sigma-2 receptors that modulate calcium and induce cytotoxicity are localized intracellularly. Soc. Neurosci. Abstr. 25:#680.19, 1708.

Bowen et al. (1997) Sigma receptor ligands modulate expression of the multidrug resistance gene in human and rodent brain tumor cell lines, Cancer Res. 38:#3206, 479.

Bowen et al. (1995a) CB-64D and CB-184: Ligands with high sigma-2 receptor affinity and subtype selectivity, Eur. J. Parmacol. 278:257-260.

Bowen et al. (1995b) Ibogaine and its congeners are sigma-2 receptor-selective ligands with moderate affinity, Eur. J. Parmacol. 279:R1-R3.

Bowen et al. (1990) Metabolites of haloperidol display preferential activity at sigma receptors compared to dopamine D-2 receptors. Eur. J. Pharmacol. 177:111-118.

Cohen, G. M. (1997) Caspases; The executioners of Apoptosis. Biochemical Journal 326:1-16.

Crawford et al. (1999) Sigma-2 receptor activation induces apoptosis in breast, prostate, and neuroblastoma cell lines, Cancer Research 40:1104, 166.

Fadok et al. (1992) Exposure of phosphatidylserine on the surface of apoptotic lymphocytes triggers specific recognition and removal of macrophages. J. Immunol. 148:2207-2216.

Hofsli et al. (1990) Reversal of Multidrug Resistance by Lipophilic Drugs, Cancer Res. 50:3997-4002.

John et al. (1996) Characterization of sigma receptor binding sites in human biopsied solid breast tumors, J. Nucl. Med. 37:267P.

Lomax et al. (1998) Characterization of p53 oligomerization domain mutations isolated from Li-Fraumeni and Li-Fraumeni like family members, Oncogene 17:643-649.

Mach et al. (1997) Sigma-2 receptors as potential biomarkers of proliferation in breast cancer, Cancer Res. 57:156-161.

Martin et al. (1990) Induction of Apoptosis (programmed cell death) in human leukemic HL-60 cells by inhibition of RNA or protein synthesis. J. Immunology 145:1859-1867.

Ogretmen et al. (1997) Expression of the mutated p53 tumor suppressor protein and its molecular and biochemical characterization in multidrug resistant MCF-7/Adt human breast cancer cells. Oncogene 14:499-506.

Ryan et al. (1998) Characterization of structural p53 mutants which show selective defects in apoptosis but not cell cycle arrest, Mol. Cell. Biol. 18:3692-3698.

Thor et al. (1998) erb B-2, p53, and Efficacy of Adjuvant Therapy in Lymph Node-positive Breast Cancer. Int. J. Cancer 90: (18) 1346-60

Vilner et al. (1995) Cytotoxic affects of sigma ligands: Sigma receptor-mediated alterations in cellular morphology and viability, J. Neurosci. 15:117-134.

Wallace-Brodeur et al. (1999) Clinical implications of p53 mutations, Cell Mol. Life Sci. 55:64-75.

We claim:

1. A method for potentiating an antineoplastic effect of a DNA-damaging antineoplastic agent on a cancer cell, comprising contacting the cancer cell with a subtoxic amount of a sigma-2 agonist and an effective amount of said DNA-damaging antineoplastic agent.

2. The method of claim 1, wherein the method potentiates the antineoplastic effect of the DNA-damaging antineoplastic agent on a non-drug resistant cancer cell.

3. The method of claim 1, wherein the cancer cell is a human cancer cell.

4. The method of claim 1, wherein the cancer cell is a drug-resistant cancer cell comprising a mutated p53 tumor-suppressor gene.

5. The method of claim 1, wherein the cancer cell is a cancer cell selected from the group consisting of a breast cancer cell, a brain cancer cell, and a prostate cancer cell.

6. The method of claim 1, wherein the sigma-2 agonist is selected from the group consisting of (+)-5,8-disubstituted morphan-7-ones and iboga alkaloids.

7. The method of claim 6 wherein the sigma-2 receptor agonist comprises: 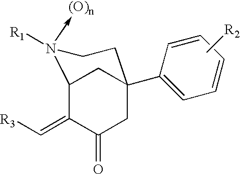
wherein:
n=0 or 1;
R₁=lower-alkyl, lower-alkenyl, cycloalkyl, lower-alkynyl, lower-alkylaryl, or hydrogen;
R₂=lower-alkyl, lower-alkoxy, lower-alkylamino, hydroxy, amino, nitro, halo, azido, or hydrogen; and
R₃=aryl, alkylaryl, lower-alkyl, cycloalkyl, lower-alkenyl, lower-alkynyl, lower-alkylaryl, lower-alkoxy, lower-alkylamino, hydrogen, or hydroxy.

8. The method of claim 7, wherein the sigma-2 ligand is selected from the group consisting of CB-184 and CB-64D.

9. The method of claim 1, wherein the sigma-2 agonist is a sigma-2 selective agonist.

10. The method of claim 9, wherein the sigma-2 selective agonist has a selectivity for the sigma-2 receptor versus the sigma-1 receptor of about 5 or greater.

11. The method of claim 1, wherein the DNA-damaging antineoplastic compound is selected from the group consisting of doxorubicin and actinomycin D.

12. The method of claim 1, wherein the DNA-damaging antineoplastic agent does not have sigma receptor activity.

13. The method of claim 1, wherein the sigma-2 agonist is haloperidol, pentazocine, or pimozide and is administered in an amount greater than about 1 mg per day.

14. The method of claim 1, wherein the sigma-2 agonist is haloperidol, pentazocine, or pimozide and is administered in an amount between about 5 mg to about 400 mg parenterally or orally per day.

15. The method of claim 1, wherein the sigma-2 agonist is haloperidol, pentazocine, or pimozide and is administered in an amount greater than about 10 mg per day.

16. The method of claim 1, wherein the sigma-2 agonist is haloperidol, pentazocine, or pimozide and is administered in an amount greater than about 25 mg per day.

17. The method of claim 1, wherein the sigma-2 agonist is haloperidol, pentazocine, or pimozide and is administered in an amount greater than about 40 mg per day.

18. The method of claim 1, wherein the sigma-2 agonist is not haloperidol, pentazocine, or pimozide.
